(19) United States
(12) Patent Application Publication
(10) Pub. No.: US 2017/0136106 A1
(43) Pub. Date: May 18, 2017

(54) BOTULINUM TOXIN ADMINISTRATION FOR TREATMENT OF NEUROGENIC DETRUSOR OVERACTIVITY ASSOCIATED URINARY INCONTINENCE

(71) Applicant: Allergan, Inc., Irvine, CA (US)

(72) Inventors: Cornelia C. Haag-Molkenteller, Irvine, CA (US); Brenda Jenkins, Mission Viejo, CA (US)

(21) Appl. No.: 15/354,263

(22) Filed: Nov. 17, 2016

Related U.S. Application Data
(60) Provisional application No. 62/256,551, filed on Nov. 17, 2015.

(51) Int. Cl. A61K 38/48; A61K 9/00

(52) U.S. Cl. CPC A61K 38/4893; C12Y 304/24069; A61K 9/0019

(57) ABSTRACT

Methods for treating urinary incontinence associated with neurogenic detrusor overactivity (NDO) and for reducing need for or frequency of clean intermittent catherization (CIC) by injecting a clostridial derivative to the bladder of a patient are disclosed.

### CROSS-REFERENCES TO RELATED APPLICATIONS

This application claims the benefit of U.S. Provisional Application No. 62/256,551 filed Nov. 17, 2015, incorporated herein by reference in its entirety.

### FIELD

The present disclosure relates to a method for treating urinary incontinence associated with neurogenic detrusor overactivity (NDO) by local administration of a clostridial neurotoxin in a patient in need thereof.

### BACKGROUND

The normal bladder has two functions, storage of urine and voiding of urine. The normal micturition process is a result of a complex network of innervation of the bladder and uretheral sphincter that ensures satisfactory bladder filling followed by timely voiding in healthy individuals. The sensations of pain and bladder fullness are carried by afferent fibers to the micturition center of the brain, triggering micturition by the coordinated relaxation of the base of the bladder and urinary sphincter.

Overactive bladder (OAB) is a condition resulting in a disruption to the normal micturition process. OAB is a symptom complex characterized by the urinary storage symptoms of urgency, with or without urgency incontinence, usually with frequency and nocturia. OAB arises without obvious pathology or due to an unknown cause, sometimes referred to as idiopathic OAB.

Incontinence is due to involuntary spontaneous contraction of the detrusor muscle during bladder filling (detrusor overactivity). Detrusor overactivity due to a relevant neurological condition, such as spinal cord injury (SCI) or multiple sclerosis (MS) is defined as neurogenic detrusor overactivity (NDO).

OAB is a condition where there is a frequent feeling of urinary urgency or needing to urinate, to a degree that it negatively affects a person's life. The frequent need to urinate may occur during the day, at night, or both. OAB is not associated with any causative infection or obvious pathological condition, and is thus by definition idiopathic. The worldwide prevalence of overactive bladder has been estimated to be between 50 and 100 million people. In the U.S. alone, 16.5% of the population (33 million) is estimated to have OAB symptoms, of which approximately 6.1% experience associated urinary incontinence.

NDO is less common than OAB and, as mentioned, is secondary to neurogenic pathology such as spinal cord injury (SCI) or multiple sclerosis (MS).

Treatment of OAB in adults and children generally begins with physiological and behavioral interventions, followed by pharmacological agents, particularly anticholinergic agents due to their direct action on the parasympathetic control of detrusor contractions. However, at doses sufficient to restore continence, side effects such as dry mouth, sedation, impaired cognition, and constipation often result in drug intolerance. NDO is also treated with pharmacological agents, such as anticholinergics, and in addition some patients use clean intermittent catheterization (CIC) to drain the bladder to manage their incontinence and high bladder pressures.

Neurotoxin therapies, in particular botulinum toxins, have been used in treatments of various medical conditions, including urological conditions such as OAB and NDO. Botulinum toxin therapy to treat bladder disorders such as OAB and NDO, is typically administered by injection across the urinary bladder wall and into the innervated muscular tissues surrounding the bladder.

Some of the common side effects associated with botulinum toxin administration into the bladder to treat bladder disorders such as OAB or NDO, include urinary tract infections, dysuria (painful voiding), and urinary retention. Urinary retention is defined as the inability to completely or partially empty the bladder. Symptoms of urinary retention include difficulty starting urination, difficulty completely emptying the bladder, weak urine stream, decreased sensation of bladder fullness, need to strain to empty bladder (valsava), and the feeling of frequently needing to empty the bladder. If left untreated, urinary retention can lead to potentially serious and life-threatening complications such as urinary tract infections, pyelonephritis, and ultimately kidney damage. A known treatment for urinary retention is clean intermittent catheterization (CIC), where a patient places a small tube into their urethra in order to drain the bladder. The risk of urinary retention and the need to perform CIC can have a negative impact on the quality of life of patients and may cause the patient to not undergo future treatments.

Thus, there is a need for an improved method of botulinum toxin administration for treatment of urological conditions such as NDO that reduces the risk for urinary retention and the need for and/or frequency of CIC, and consequently has an improved safety profile and enhanced efficacy.

### SUMMARY OF THE INVENTION

Aspects of the present disclosure provide a method for treating urinary incontinence associated with NDO in a patient in need thereof, the method comprising locally administering to the bladder of the patient a composition comprising an amount of less than 200 Units of a clostridial derivative.

In some embodiments, the method comprises locally administering a composition comprising a therapeutically effective amount of a clostridial derivative to the bladder of the patient. In some embodiments, the method comprises administering the clostridial derivative to the detrusor muscle.

In some embodiments, the amount is less than 200 Units of a botulinum toxin type A. In at least one embodiment, the method comprises administering about 100 Units of a botulinum toxin type A.

In some embodiments, the method comprises administering the amount in less than about 30 injection sites. In at least one embodiment, the method comprises administering the amount in about 25 injection sites. In at least one embodiment, the method comprises administering the therapeutically effective amount in about 20 injection sites. In at least one embodiment, the method comprises administering the therapeutically effective amount in about 15 injection sites.

In some embodiments, the clostridial derivative is a botulinum toxin. In some embodiments, the clostridial derivative is a botulinum toxin type A. In some embodiments, the clostridial derivative is onabotulinumtoxin A. In some embodiments, the clostridial derivative is abobotulinumtoxin A.

In some embodiments, the method comprises locally administering to a patient with urinary incontinence due to NDO resulting from multiple sclerosis (MS) or spinal cord injury (SCI). In some embodiments, the locally administering is to a patient having urinary incontinence due to NDO resulting from MS or SCI and who is not using CIC. In some embodiments, the locally administering is to a patient who had an inadequate response to or is intolerant of anticholinergic medication. In another embodiment, the locally administering is to a patient having urinary incontinence due to NDO resulting from MS or SCI and who had an inadequate response to or is intolerant of anticholinergic medication.

In another aspect, the present disclosure provides a method for reducing or preventing the risk for urinary retention associated with neurogenic detrusor overactivity treatment using a clostridial derivative in a patient, the method comprising locally administering a composition comprising an amount of less than 200 Units of the clostridial derivative to the detrusor. In some embodiments, the amount is less than 200 Units of a botulinum toxin type A. In at least one embodiment, the method comprises administering about 100 Units of a botulinum toxin type A. In some embodiments, the method comprises administering the amount in less than about 30 injection sites. In at least one embodiment, the method comprises administering the amount in about 20 injection sites. In some embodiments, the clostridial derivative is a botulinum toxin. In some embodiments, the clostridial derivative is a botulinum toxin type A.

In some embodiments, the method further comprises selecting a patient with urinary incontinence due to NDO resulting from multiple sclerosis (MS). In some embodiments, the locally administering is to a patient having urinary incontinence due to NDO resulting from MS or SCI and who is not using CIC. In some embodiments, the locally administering is to a patient who had an inadequate response to or is intolerant of anticholinergic medication. In another embodiment, the locally administering is to a patient having urinary incontinence due to NDO resulting from MS or SCI and who had an inadequate response to or is intolerant of anticholinergic medication.

In another aspect, the present disclosure provides a method for reducing the need for clean intermittent catherization (CIC) associated with neurogenic detrusor overactivity treatment using a clostridial derivative in a patient. The method comprising locally administering to the bladder of the patient a composition comprising an amount of less than 200 Units of the clostridial derivative.

In some embodiments, the reduced need for CIC corresponds to a 20%, 30%, 40% or 50% reduction in frequency of CIC performed by the patient, where the percent reduction is relative to patients treated with 200 U of the clostridial derivative or treated with greater than about 200 U of the clostridial derivative.

In some embodiments, the amount is less than 200 Units of a botulinum toxin type A. In at least one embodiment, the method comprises administering about 100 Units of a botulinum toxin type A.

In some embodiments, the method comprises administering the therapeutically effective amount in less than about 30 injection sites. In at least one embodiment, the method comprises administering the therapeutically effective amount in about 20 injection sites.

In some embodiments, the clostridial derivative is a botulinum toxin. In some embodiments, the clostridial derivative is a botulinum toxin type A.

In some embodiments, the locally administering is to a patient with urinary incontinence due to NDO resulting from multiple sclerosis (MS). In some embodiments, the locally administering is to a patient having urinary incontinence due to NDO resulting from MS or SCI and who is not using CIC. In some embodiments, the locally administering is to a patient who had an inadequate response to or is intolerant of anticholinergic medication. In another embodiment, the locally administering is to a patient having urinary incontinence due to NDO resulting from MS or SCI and who had an inadequate response to or is intolerant of anticholinergic medication.

In another aspect, by alleviating one or more adverse events or side effects associated with clostridial treatment of overactive bladder or detrusor overactivity in a patient in need thereof, the present method provides functional improvement and thus improves the quality of life for the patient.

### BRIEF DESCRIPTION OF THE DRAWINGS

The following drawings are presented to illustrate aspects and features of embodiments of the present invention.

FIG. 1 shows an exemplary result of the change from baseline in Urinary Incontinence by administration of an injection paradigm in accordance with aspects of the present disclosure;

FIG. 2 shows a Urinary Incontinence Responder Analysis by administration of the injection paradigm in accordance with aspects of the present disclosure;

FIGS. 3A and 3B show a change from Baseline in UDS Endpoints in Mean Cystometric Capacity (MCC) (FIG. 3A) and Maximum Detrusor Pressure (MDP) (FIG. 3B) by administration of the injection paradigm in accordance with aspects of the present disclosure;

FIG. 4 shows a change from baseline to I-QOL Total score;

FIG. 5 shows a change from baseline to I-QOL Total score at week 6 and includes a comparison of CIC versus No CIC;

FIG. 6 shows a duration of effect;

FIGS. 7A and 7B show a change from Baseline in Urinary Incontinence Episodes/Day in a 20 site injection paradigm (FIG. 7A) versus a 30 injection site injection paradigm (FIG. 7B); and

FIGS. 8A and 8B show a comparison in Duration of Effect between a 20 site injection paradigm (upper panel, FIG. 8A) versus 30 injection site injection paradigm (lower panel, FIG. 8B).

### DETAILED DESCRIPTION OF THE PREFERRED EMBODIMENTS

Botulinum neurotoxins (BoNTs) such as, for example, BoNT/A, BoNT/B, etc., act on the nervous system by blocking the release of neurosecretory substances such as neurotransmitters. The action of BoNT is initiated by its binding to a receptor molecule on the cell surface, and then the toxin-receptor complex undergoes endocytosis. Once inside the cell, BoNT cleaves exocytotic specific proteins responsible for neurotransmitter docking and release from the cell, known as the SNARE proteins (soluble N-ethylmaleimide-sensitive factor attachment protein receptor). The resulting transient chemodenervation has been utilized medically to block motor neurotransmission at the neuromuscular junction leading to a variety of therapeutic applications.

The following definitions apply herein:

“About” or “approximately” as used herein means within an acceptable error range for the particular value as determined by one of ordinary skill in the art, which will depend in part on how the value is measured or determined, (i.e., the limitations of the measurement system). For example, “about” can mean within 1 or more than 1 standard deviations, per practice in the art. Where particular values are described in the application and claims, unless otherwise stated, the term “about” means within an acceptable error range for the particular value.

“Administration”, or “to administer” means the step of giving (i.e. administering) a botulinum toxin to a subject, or alternatively a subject receiving a pharmaceutical composition.

“Alleviating” means a reduction in the occurrence of a pain or other symptoms associated with bladder overactivity. Thus, alleviating includes some reduction, significant reduction, near total reduction, and total reduction. An alleviating effect may not appear clinically for between 1 to 7 days after administration of a clostridial derivative to a patient or sometimes thereafter.

“Botulinum toxin” means a neurotoxin produced by Clostridium botulinum, as well as a botulinum toxin (or the light chain or the heavy chain thereof) made recombinantly by a non-Clostridial species. The term “botulinum toxin”, as used herein, encompasses the botulinum toxin serotypes A, B, C, D, E, F and G, and their subtypes and any other types of subtypes thereof, or any re-engineered proteins, analogs, derivatives, homologs, parts, sub-parts, variants, or versions, in each case, of any of the foregoing. “Botulinum toxin”, as used herein, also encompasses a “modified botulinum toxin”. Further “botulinum toxin” as used herein also encompasses a botulinum toxin complex, (for example, the 300, 600 and 900 kDa complexes), as well as the neurotoxic component of the botulinum toxin (150 kDa) that is unassociated with the complex proteins.

“Clostridial derivative” refers to a molecule which contains any part of a clostridial toxin. As used herein, the term “clostridial derivative” encompasses native or recombinant neurotoxins, recombinant modified toxins, fragments thereof, a Targeted vesicular Exocytosis Modulator (TEM), or combinations thereof.

“Clostridial toxin” refers to any toxin produced by a Clostridial toxin strain that can execute the overall cellular mechanism whereby a Clostridial toxin intoxicates a cell and encompasses the binding of a Clostridial toxin to a low or high affinity Clostridial toxin receptor, the internalization of the toxin/receptor complex, the translocation of the Clostridial toxin light chain into the cytoplasm and the enzymatic modification of a Clostridial toxin substrate.

“Effective amount” as applied to the biologically active ingredient means that amount of the ingredient which is generally sufficient to induce a desired change in the subject. For example, where the desired effect is a reduction in calculi formation, an effective amount of the ingredient is that amount which causes at least a substantial reduction of bladder overactivity and associated symptoms, and without resulting in significant toxicity.

“Implant” means a controlled release (e.g., pulsatile or continuous) composition or drug delivery system. The implant can be, for example, injected, inserted or implanted into a human body.

“Local administration” or “locally administering” means administration of a pharmaceutical agent to or to the vicinity of a muscle or a subdermal location in a patient by a non-systemic route. Thus, local administration excludes systemic routes of administration, such as intravenous or oral administration.

“Peripheral administration” means administration to a location away from a symptomatic location, as opposed to a local administration.

“TEMs”, abbreviated for Targeted Exocytosis Modulators are retargeted endopeptidases that direct the catalytic activity of the light chain to specific types of neuronal cells or to target cells that were not affected by botulinum toxins expanding the beneficial clinical effect of inhibition of exocytosis in several human diseases.

“Treating” or “treatment” means to alleviate (or to eliminate) at least one symptom (such as, for example, hip and groin pain), either temporarily or permanently.

“Therapeutically effective amount” refers to an amount sufficient to achieve a desired therapeutic effect.

Aspects of the present disclosure provide a method for treating urinary incontinence associated with NDO in a patient in need thereof, the method comprising locally administering a composition comprising an amount of a clostridial derivative to the bladder. In some embodiments, the method comprising locally administering a composition comprising a therapeutically effective amount of a clostridial derivative to the bladder. In some embodiments, the method comprises locally administering a composition comprising a therapeutically effective amount of a clostridial derivative to the bladder of the patient. In some embodiments, the method comprises administering the clostridial derivative to the detrusor muscle.

In some embodiments, the therapeutically effective amount is less than 200 Units of a botulinum toxin type A. In at least one embodiment, the method comprises administering about 100 Units of a botulinum toxin type A. In some embodiments, the method comprises administering the therapeutically effective amount in about 30 injection sites. In some embodiments, the method comprises administering the therapeutically effective amount in less than about 30 injection sites. In at least one embodiment, the method comprises administering the therapeutically effective amount in about 25 injection sites. In at least one embodiment, the method comprises administering the therapeutically effective amount in about 20 injection sites. In at least one embodiment, the method comprises administering the therapeutically effective amount in about 15 injection sites. In some embodiments, each of the injections is at different injection site. In alternative embodiments, at least some of the injections are at the same injection site (i.e. the number of injection sites is less than the number of injections).

In some embodiments, the clostridial derivative is a botulinum toxin. In some embodiments, the clostridial derivative is a botulinum toxin type A. In some embodiments, the clostridial derivative is onabotulinumtoxinA. In some embodiments, the clostridial derivative is abobotulinumtoxinA.

The FDA has approved an injection paradigm of 200 Units of a botulinum toxin type A in the detrusor muscle of the bladder to treat urinary incontinence associated with NDO (See, Prescribing Information for BOTOX®). One of the potential adverse drug reactions of botulinum toxin type A treatment is urinary retention due to the relaxation of the detrusor muscle so the patient has difficulty emptying their bladder fully and they may need to use CIC temporarily to empty their bladder.

Among other benefits, the present invention provides a method that reduces the risk for urinary retention and/or reduces the need for CIC. In certain embodiments the method substantially reduces the risk for urinary retention and the frequency of CIC.

In another aspect, the present invention provides a method for reducing or preventing the risk of urinary retention associated with neurogenic detrusor overactivity treatment using a clostridial derivative in a patient, the method comprising locally administering a composition comprising an amount of the clostridial derivative to the detrusor. In some embodiments, the amount is less than 200 Units of a botulinum toxin type A. In at least one embodiment, the method comprises administering about 100 Units of a botulinum toxin type A. In some embodiments, the method comprises administering the therapeutically effective amount in about 30 injection sites. In some embodiments, the method comprises administering the amount in less than about 30 injection sites. In at least one embodiment, the method comprises administering the amount in about 25 injection sites. In at least one embodiment, the method comprises administering the therapeutically effective amount in about 20 injection sites. In at least one embodiment, the method comprises administering the therapeutically effective amount in about 15 injection sites. In some embodiments, each of the injections is at a different injection site. In alternative embodiments, at least some of the injections are at the same injection site (i.e. the number of injection sites is less than the number of injections). In some embodiments, the clostridial derivative is a botulinum toxin. In some embodiments, the clostridial derivative is a botulinum toxin type A.

In some embodiments, the method further comprises selecting a patient with urinary incontinence due to NDO resulting from multiple sclerosis (MS) or from spinal cord injury (SCI). In some embodiments, the selecting step further comprises identifying a patient having urinary incontinence due to NDO resulting from MS or SCI and who is not using CIC. In some embodiments, the selecting step further comprises identifying a patient who had an inadequate response to or is intolerant to anticholinergic medication.

In another aspect, the present invention provides a method for reducing the need for or frequency of clean intermittent catherization (CIC) associated with neurogenic detrusor overactivity treatment using a clostridial derivative in a patient. The method comprising locally administering a composition comprising a therapeutically effective amount of the clostridial derivative to the bladder. In one embodiment, the frequency of CIC corresponds to a 20%, 30%, 40% or 50% reduction in frequency of CIC performed by the patient over a given time period, such as daily, weekly or over a six week period, where the percent reduction is relative to patients treated with 200 U of the clostridial derivative, patients treated with greater than about 200 U of the clostridial derivative, or relative to the patient prior to treatment with less than about 200 U of the clostridial derivative.

In some embodiments, the therapeutically effective amount is less than 200 Units of a botulinum toxin type A. at least one embodiment, the method comprises administering about 100 Units of a botulinum toxin type A. In some embodiments, the method comprises administering the therapeutically effective amount in less than about 30 injection sites. In some embodiments, the method comprises administering the therapeutically effective amount in less than about 30 injection sites. In one embodiment, the method comprises administering the therapeutically effective amount in about 25 injection sites. In at least one embodiment, the method comprises administering the therapeutically effective amount in about 20 injection sites. In at least one embodiment, the method comprises administering the therapeutically effective amount in about 15 injection sites.

In some embodiments, the clostridial derivative is a botulinum toxin. In some embodiments, the clostridial derivative is a botulinum toxin type A.

In some embodiments, the method further comprises selecting a patient with urinary incontinence due to NDO resulting from MS or SCI. In some embodiments, the selecting step further comprises identifying a patient having urinary incontinence due to NDO resulting from MS or SCI and who is not using CIC. In some embodiments, the selecting step further comprises identifying a patient who had an inadequate response to or are intolerant to anticholinergic medication.

In some embodiments, the composition is administered by injections, including intramuscular injections and/or non-intramuscular injections.

In some embodiments, the clostridial derivative includes a native, recombinant clostridial toxin, recombinant modified toxin, fragments thereof, TEMs, or combinations thereof. In some embodiments, the clostridial derivative is a botulinum toxin. In some embodiments, the botulinum toxin can be a botulinum toxin type A, type B, type C₁, type D, type E, type F, or type G, or any combination thereof. The botulinum neurotoxin can be a recombinantly made botulinum neurotoxins, such as botulinum toxins produced by E. coli. In alternative embodiments, the clostridial derivative is a TEM.

In some embodiments, the botulinum neurotoxin can be a modified neurotoxin, that is a botulinum neurotoxin which has at least one of its amino acids deleted, modified or replaced, as compared to a native toxin, or the modified botulinum neurotoxin can be a recombinant produced botulinum neurotoxin or a derivative or fragment thereof. In certain embodiments, the modified toxin has an altered cell targeting capability for a neuronal or non-neuronal cell of interest. This altered capability is achieved by replacing the naturally-occurring targeting domain of a botulinum toxin with a targeting domain showing a selective binding activity for a non-botulinum toxin receptor present in a non-botulinum toxin target cell. Such modifications to a targeting domain result in a modified toxin that is able to selectively bind to a non-botulinum toxin receptor (target receptor) present on a non-botulinum toxin target cell (re-targeted). A modified botulinum toxin with a targeting activity for a non-botulinum toxin target cell can bind to a receptor present on the non-botulinum toxin target cell, translocate into the cytoplasm, and exert its proteolytic effect on the SNARE complex of the target cell. In essence, a botulinum toxin light chain comprising an enzymatic domain is intracellularly delivered to any desired cell by selecting the appropriate targeting domain.

The clostridial derivative, such as a botulinum toxin, for use according to the present methods can be stored in lyophilized, vacuum dried form in containers under vacuum pressure or as stable liquids. Prior to lyophilization the botulinum toxin can be combined with pharmaceutically acceptable excipients, stabilizers and/or carriers, such as, for example, albumin, or the like. In embodiments containing albumin, the albumin can be, for example, human serum albumin, or the like. The lyophilized material can be reconstituted with a suitable liquid such as, for example, saline, water, or the like to create a solution or composition containing the botulinum toxin to be administered to the patient.

In some embodiments, the clostridial derivative is provided in a controlled release system comprising a polymeric matrix encapsulating the clostridial derivative, wherein fractional amount of the clostridial derivative is released from the polymeric matrix over a prolonged period of time in a controlled manner. Controlled release neurotoxin systems have been disclosed for example in U.S. Pat. Nos. 6,585,993; 6,585,993; 6,306,423 and 6,312,708, each of which is hereby incorporated by reference in its entirety.

The therapeutically effective amount of the botulinum toxin, in the present method can vary according to the potency of a particular botulinum toxin, as commercially available Botulinum toxin formulations do not have equivalent potency units. It has been reported that one Unit of BOTOX® (onabotulinumA), a botulinum toxin type A available from Allergan, Inc., has a potency Unit that is approximately equal to 3 to 5 Units of DYSPORT® (abobotulinumtoxinA), also a botulinum toxin type A available from Ipsen Pharmaceuticals. MYOBLOC®, a botulinum toxin type B available from Elan, has been reported to have a much lower potency Unit relative to BOTOX®. In some embodiments, the botulinum neurotoxin can be a pure toxin, devoid of complexing proteins, such as XEOMIN® (incobotulinumtoxinA). One Unit of incobotulinumtoxinA has been reported to have potency approximately equivalent to one Unit of onabotulinumtoxinA. Thus, the quantity of toxin administered and the frequency of its administration will be at the discretion of the physician responsible for the treatment and will be commensurate with questions of safety and the effects produced by a particular toxin formulation.

In some embodiments, the present method comprises administering a composition comprising between about 10-500 Units of a botulinum toxin type A to the detrusor. In some embodiments, the botulinum toxin type A is onabotulinumtoxinA, commercially available under the trade name BOTOX®. In some embodiments, the present method comprises administering a composition comprising less than 200 Units of onabotulinumtoxinA to the detrusor. In one specific embodiment, the present method comprises administering a composition comprising about 150 Units or about 100 Units of onabotulinumtoxinA to the detrusor. In some embodiments, the composition is administered to the target site of the bladder or its vicinity, e.g. the detrusor. In certain embodiments, the dosage can range from about 10 Units to less than about 200 Units per treatment. In another embodiment, the dosage ranges from about 10 Units to about 190 Units, from about 10 Units to about 175 Units, from about 10 Units to about 150 Units or from about 10 Units to about 100 U.

In some embodiments, the pharmaceutical composition can be administered at multiple sites, ranging from 1 site up to about 50 sites. In some embodiments, each of the injections is at a different injection site. In alternative embodiments, at least some of the injections are at the same each injection site (i.e. the number of injection sites is less than the number of injections). In some embodiments, the composition is administered at about 30 injection sites. In some embodiments, the composition is administered at less than 30 sites. In some embodiments, the composition is administered at about 25 injection sites. In some embodiments, the composition is administered at about 20 sites. In some embodiments, the composition is administered at about 15 sites.

In at least one embodiment, the method comprises administering about 100 Units of botulinum toxin A to about 20 injections sites in the detrusor. In at least one embodiment, the method comprises administering about 100 Units of botulinum toxin A to about 30 injections sites in the detrusor. In some embodiments, if the neurotoxin is abobotulinumtoxinA, the method comprises administering about 200 Units to about 750 Units of the abobotulinumtoxinA to the detrusor. In some embodiments, the method comprises administering about 300 Units of the abobotulinumtoxinA to the detrusor. In some embodiments, the method comprises administering about 350 Units of the abobotulinumtoxinA to the detrusor. In some embodiments, the method comprises administering about 400 Units of the abobotulinumtoxinA to the detrusor. In some embodiments, the method comprises administering about 450 Units of the abobotulinumtoxinA to the detrusor. In some embodiments, the method comprises administering about 500 Units of the abobotulinumtoxinA to the detrusor. In some embodiments, the method comprises administering about 550 Units of the abobotulinumtoxinA to the detrusor. In some embodiments, the method comprises administering about 600 Units of the abobotulinumtoxinA to the detrusor. In some embodiments, the dose range of about 200 Units to 750 Units of the abobotulinumtoxinA is administered in about 10 to about 50 injection sites. In some embodiments, the dose range of about 200 Units to 750 Units of the abobotulinumtoxinA is administered in about 15 to 30 injection sites. In at least one embodiment, the method comprises administering about 300 Units of the abobotulinumtoxinA to about 15 injection sites in the detrusor. In at least one embodiment, the method comprises administering about 300 Units of the abobotulinumtoxinA to about 20 injection sites in the detrusor. In at least one embodiment, the method comprises administering about 300 Units of the abobotulinumtoxinA to about 25 injection sites in the detrusor. In at least one embodiment, the method comprises administering about 300 Units of the abobotulinumtoxinA to about 30 injection sites in the detrusor. In some embodiments, if the neurotoxin is botulinum toxin type B, the dosage is approximately 50 times greater than the functionally equivalent dosage of botulinum toxin type A.

The treatment effects of the clostridial derivative can persist for between about 1 month and 5 years or from about 5 months to about 5 years or from about 1 year to about 4 years. Administration can be repeated as necessary. Botulinum toxin type A administered into or near detrusor muscle tissue can produce flaccid paralysis at target site muscles for between about 6 months to about 18 months. However, increased efficacy of the treatment using botulinum toxin type A occurs when the toxin is administered according to the disclosed method at about 6-12 month intervals, preferably at about a 9 month interval.

A method within the scope of the present disclosure can provide improved patient function. “Improved patient function” can be defined as an improvement measured by factors such as a reduced pain, increased ambulation, healthier attitude, more varied lifestyle and/or healing permitted by normal muscle tone and function. Improved patient function may be measured with an improved quality of life (QOL) or Health-Related Quality of Life (HRQL). Scores obtained can be compared to published values available for various general and patient populations.

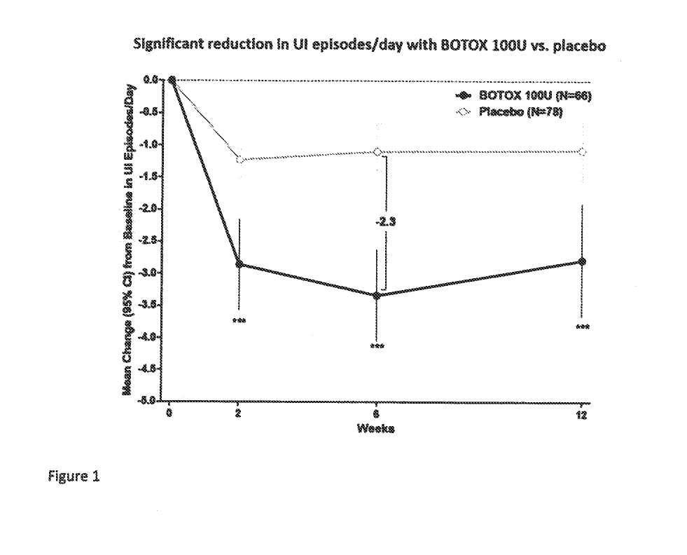

In the examples detailed below, subjects with_urinary incontinence due to NDO resulting from MS, who were not catheterizing at baseline and had an inadequate response to or are intolerant to anticholinergic medication were enrolled for treatment with 100 Units of onabotulinumtoxinA. As detailed in Example 1, primary and secondary efficacy endpoints were evaluated to determine whether the dose was effective in treating urinary incontinence, reducing the frequency of CIC, and other measures. Results from the study are set forth in FIGS. 1-6 and are discussed more fully in Example 1, and briefly here.

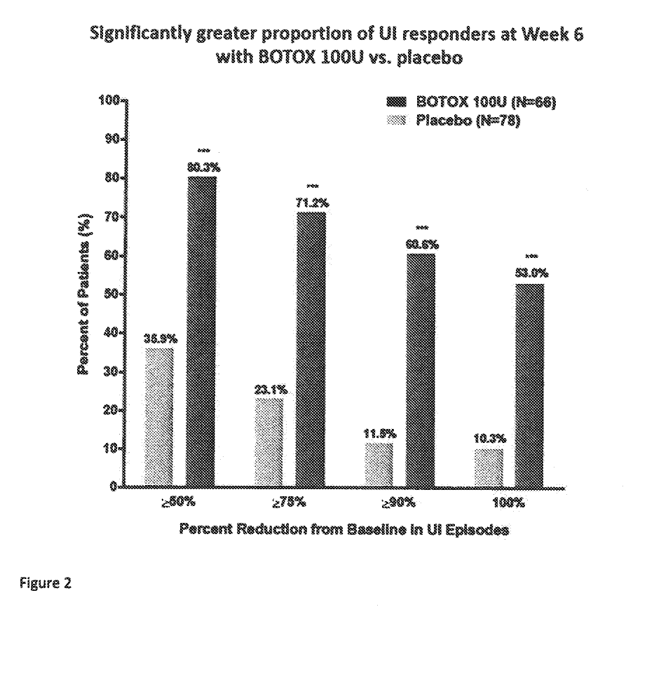

Treatment with onabotulinumtoxinA versus placebo resulted in statistically significant and clinically meaningful improvements from baseline in all primary and secondary efficacy endpoints (incontinence, urodynamic parameters and QOL), which were evident during the first 12 weeks of treatment. For example, treatment with 100 U of onabotulinumtoxinA provided a statistically significant reduction in daily urinary incontinence episodes compared with placebo, as seen in FIG. 1 and FIG. 2. Accordingly, in one embodiment, a method for treating urinary incontinence by locally administering less than about 200 Units of a clostridial derivative, such as a botulinum toxin, to a subject suffering from urinary incontinence associated with neurogenic detrusor overactivity is provided. In one embodiment, the local administration to the bladder provides a two-fold reduction in daily urinary incontinence episodes relative to the daily urinary incontinence episodes prior to the local administration, or relative to a patient population with NDO and not treated with clostridial derivative, such as a botulinum toxin. Alternatively, the local administration to the bladder provides about 40 to 80% reduction in daily urinary incontinence episodes relative to the daily urinary incontinence episodes prior to locally administering or relative to a patient population with NDO and not treated with clostridial derivative, such as a botulinum toxin.

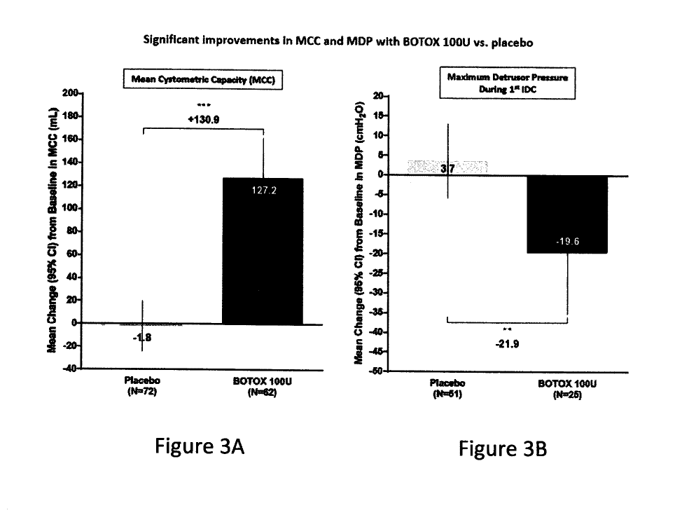

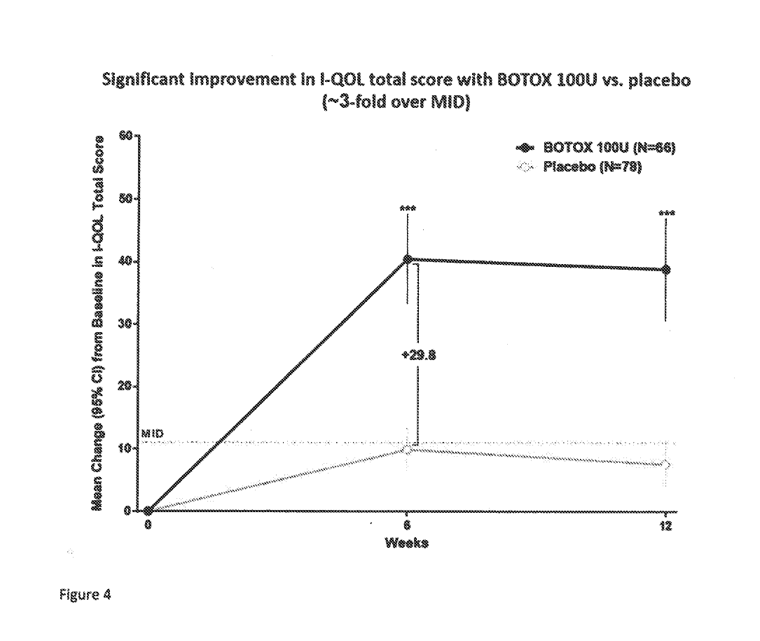

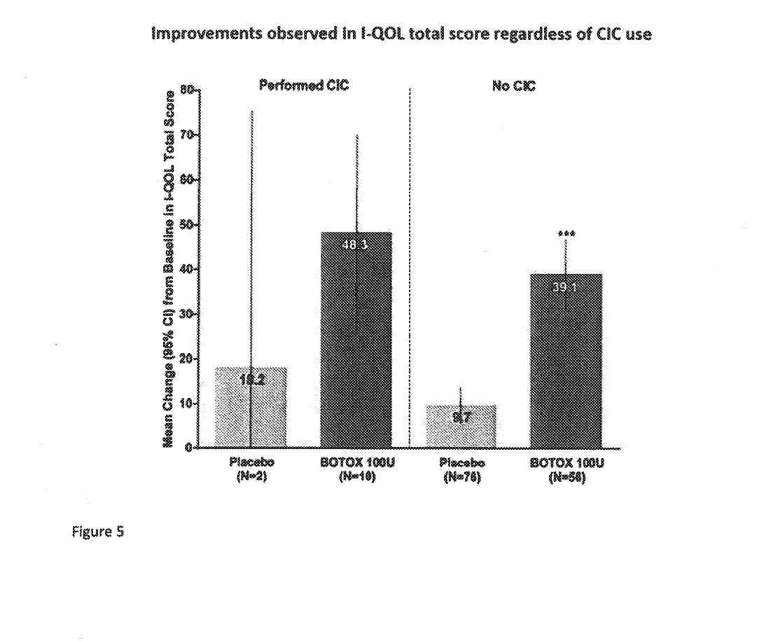

A statistically significant (p<0.001) longer duration of effect was observed in subjects treated with less than 200 U of onabotulinumtoxinA compared with placebo, based on the median time to request retreatment. Significant improvements in bladder function were also observed following onabotulinumtoxinA treatment at less than 200 U, which were reflected by significant changes in key urodynamic parameters, such as increased MCC, VPmaxIDC, greater volume per void, and reduced MDP during first IDC as well as reduced MDP during the storage phase (FIGS. 3A-3B). Consistent with these effects, and as seen in FIGS. 4-5, quality of life, as measured by I-QOL, was significantly improved in patients treated with less than 200 U of onabotulinumtoxinA. Furthermore, a significant increase in the I-QOL total summary score at week 6 was observed with less than 200 U of onabotulinumtoxinA treatment relative to placebo, with less than 200 U of onabotulinumtoxinA demonstrating an approximately 3 fold increase over the minimally important difference (MID) and placebo. The onabotulinumtoxinA was safe and well tolerated in this patient population, with no new safety signals observed during the study. The most commonly reported adverse events were urinary tract infection, bacteriuria, urinary retention, and residual urine volume, which were mostly mild or moderate in intensity.

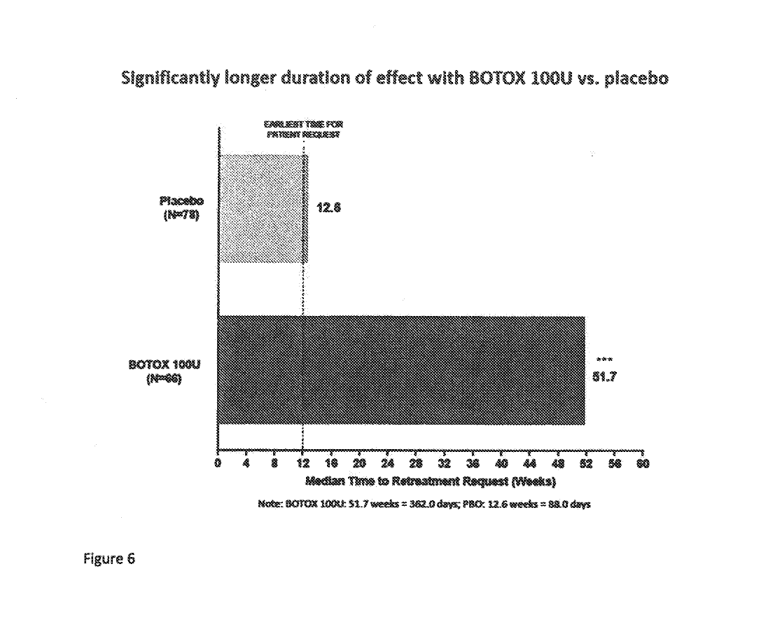

FIG. 6 shows the duration of effect of treatment with botulinum toxin compared to placebo treatment. The median time to request retreatment for the subjects treated with 100 U of botulinum toxin was about 51.7 weeks, compared to 12.6 weeks for placebo treated patients. Consequently, a significantly lower proportion of patients treated with botulinum toxin requested retreatment compared with patients treated with placebo.

The incidence of CIC initiation in this MS population of subjects in the study of Example 1 was relatively low at 15.2% in the patients treated via local administration with a composition comprising less than 200 U of onabotulinumtoxinA and 2.6% in the patients treated with placebo. This is in contrast to previously reported incidence of CIC initiation of 31.4% in patients treated with 200 U onabotulinumtoxinA and 4.5% in patients treated with placebo (Ginsberg D. et al., Adv. Ther., 2013, 30(9):819-833). Accordingly, in one embodiment, a method for reducing clean intermittent catherization (CIC) associated with treatment of neurogenic detrusor overactivity (NDO) with a botulinum toxin in a patient is provided. By locally administering to the bladder of the patient a composition comprising less than 200 Units of a botulinum toxin, the frequency of CIC is reduced by about 30%, 40% or 50%, relative to patients treated with 200 U of the botulinum toxin or treated with greater than about 200 U of the botulinum toxin.

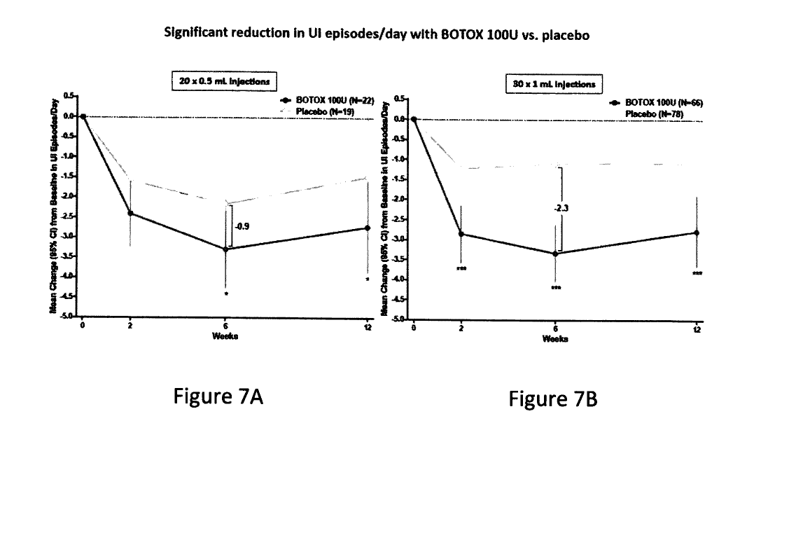

In a sub-study of the study of Example 1, described in Example 2, subjects with multiple sclerosis and intolerant to anticholinergic medication diagnosed with NDO were treated with 100 U onabotulinumtoxinA provided as 20 injections of 0.5 mL. Treatment with this injection paradigm was compared with the results for subjects treated with 100 U onabotulinumtoxinA provided as 30 injections of 1 mL in the study of Example 1. Safety and efficacy results were similar for the subjects treated with 100 U onabotulinumtoxinA provided as 20 injections of 0.5 mL compared with the results for subjects treated with 100 U onabotulinumtoxinA provided as 30 injections of 1 mL observed in the study of Example 1, as shown by the data in Table 1 set forth in Example 2 below. A reduction in urinary incontinence episodes per day was also observed in the subjects treated with 100 U onabotulinumtoxinA administered according to either dosing paradigm, as seen in FIGS. 7A-7B. In one embodiment, local administration of a botulinum toxin A to the bladder of about 100 Units injected at about 15, 20, 25 or 30 injection sites provides about 40-80% reduction in daily urinary incontinence episodes relative to daily urinary incontinence episodes before treatment. In other embodiments, a composition comprising about 100 Units of a botulinum toxin A is administered to the bladder of the patient, wherein the administration is by injection to about 20 injection sites. In another embodiment, a composition comprising about 100 Units of a botulinum toxin A is administered to the bladder of the patient, wherein the administration is by injection to about 30 injection sites.

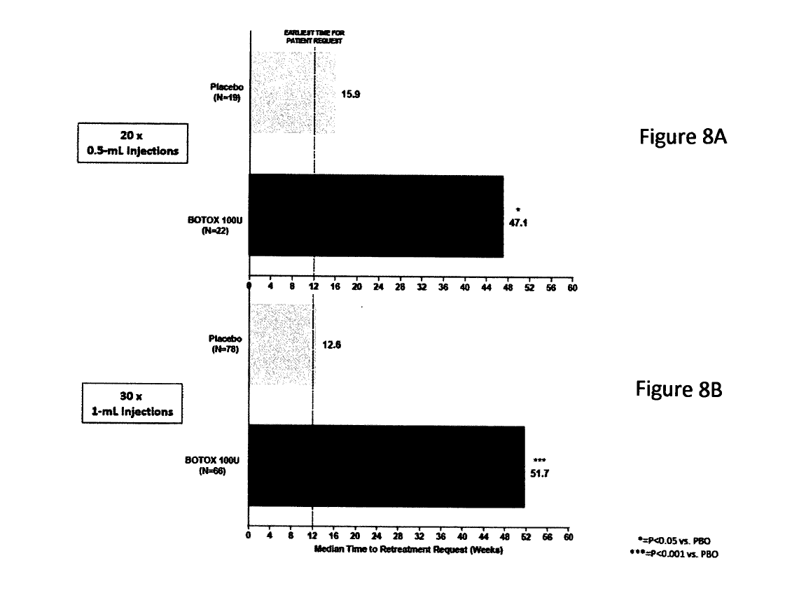

FIG. 8 shows the duration of effect of treatment with botulinum toxin administered according to the two injection paradigms compared to placebo treatment. The median time to request retreatment for the subjects treated with 100 U of botulinum toxin administered over 20 injection sites was 47.1 weeks, comparted to 15.9 weeks for placebo treated patients. The median time to request retreatment for the subjects treated with 100 U of botulinum toxin administered over 30 injection sites was about 51.7 weeks, compared to 12.6 weeks for placebo treated patients. Consequently, a significantly lower proportion of patients treated with botulinum toxin requested retreatment compared with patients treated with placebo.

Accordingly, and based on the evidence provided herein, a method for the treatment of urinary incontinence due to detrusor overactivity associated with a neurologic condition (e.g., SCI, MS) in adults who have an inadequate response to or are intolerant of an anticholinergic medication is provided. Also contemplated in a method for treating neurogenic detrusor overactivity with urinary incontinence due to subcervical spinal cord injury (traumatic or non-traumatic) or multiple sclerosis. Also contemplated in a method for treating urinary incontinence in adults with neurogenic detrusor overactivity resulting from neurogenic bladder due to stable sub-cervical spinal cord injury or with multiple sclerosis. A method for the treatment of urinary incontinence associated with neurogenic detrusor overactivity not controlled with an anticholinergic treatment in patients with spinal cord injury, patients with muscular sclerosis and using clean intermittent catheterization is also provided. Method for the treatment of neurogenic detrusor overactivity with urinary incontinence due to subcervical spinal cord injury (traumatic or non-traumatic) or multiple sclerosis are provided. The management of urinary incontinence due to neurogenic detrusor overactivity associated with a neurologic condition (such as spinal cord injury, multiple sclerosis) in adults is also contemplated, as is the treatment of urinary incontinence due to detrusor overactivity associated with spinal cord lesion in adults not controlled adequately by anticholinergic agents. In these methods, the treatment comprises administering to the bladder a composition comprising an amount of less than 200 Units a clostridial derivative.

The following non-limiting examples provide those of ordinary skill in the art with specific preferred methods to treat urological dysfunctions such as NDO within the scope of the present disclosure, and it is not intended to limit the scope of the invention. In the following examples various modes of non-systemic administration of a botulinum neurotoxin can be carried out. For example, by intramuscular injection, non-intramuscular injection or by implantation of a controlled release implant.

### EXAMPLES

The following non-limiting examples provide those of ordinary skill in the art with specific preferred methods to treat conditions within the scope of embodiments of the present invention and are not intended to limit the scope of the invention.

### Example 1

### Treatment of NDO with 100 Units of OnabotulinumtoxinA

This was a multicenter, double-blind, randomized, placebo-controlled, parallel-group study designed to assess the safety and efficacy of 100 Units (U) onabotulinumtoxinA) (BOTOX® compared to placebo for the treatment of urinary incontinence due to NDO. The 100 U of onabotulinumtoxinA or the placebo were administered as 30 injections of 1 mL each.

Patient Population:

Patients with urinary incontinence due to NDO resulting from MS, who were not catheterizing at baseline and had an inadequate response to or are intolerant to anticholinergic medication were selected. Patient subpopulations included non-catheterizing MS patients, patients not responding or intolerant to anticholinergic medication. Excluded from the study were patients currently using clean intermittent catheterization (CIC) (at any frequency), or an indwelling catheter, to manage their urinary incontinence were excluded from the study.

Efficacy and Safety Measurements:

The primary efficacy measure was number of daily urinary incontinence episodes. The secondary efficacy measures were maximum cystometric capacity (MCC), maximum detrusor pressure during the first involuntary detrusor contraction (PmaxIDC), and Incontinence Quality of Life Instrument (I-QOL) total summary score. Other efficacy measures included proportion of patients who had ≧50% (definition of responder), ≧75%, ≧90%, and 100% reduction (incontinence-free/dry) from study baseline in daily urinary incontinence episodes as recorded in patient bladder diary, presence/absence of involuntary detrusor contraction (IDC) and if present, volume at first involuntary detrusor contraction (VPmaxIDC), maximum detrusor pressure during the storage phase (Pdetmax), bladder diary parameters (volume voided per voiding episode [voluntary and catheterization], number of micturition episodes, number of urgency episodes, number of nocturia episodes), duration of effect (time to patient request for retreatment and proportion of patients requesting retreatment), time to patient qualification for retreatment, and proportion of patients qualifying for retreatment during the study.

Health Outcomes:

I-QOL total summary score and individual domain scores and Overactive Bladder Patient Satisfaction with Treatment Questionnaire (OAB-PSTQ) score.

Safety:

The safety measures were adverse events and serious adverse events; physical examination; vital signs; kidney and bladder ultrasound; PVR urine volume; use of CIC (proportion of patients, frequency, and duration); MS exacerbation rates and MS exacerbation annualized rates (total number of exacerbation events divided by the total exposure time); immunogenicity testing; hematology and nonfasting clinical chemistry; urine dipstick reagent strip test; urinalysis (with urine culture and sensitivity, as applicable); UTI rates by antibiotic prophylaxis; pregnancy test for women of childbearing potential; EDSS (at screening only); prostate-specific antigen (PSA) for male patients (at screening only); urine cytology (at screening only); concomitant medications; concurrent procedures.

Summary of Results:

To simplify the discussion of results, the patients treated with 100 U BOTOX is referred to as the BOTOX group, and those treated with placebo are referred to as the placebo group.

Efficacy:

Statistically significant reductions in urinary incontinence episodes per day were observed at weeks 2, 6 (the primary endpoint), and 12 compared with placebo. At week 6, the mean (SD) percentage change from baseline was −3.3 (2.88) in the BOTOX group compared with −1.1 (2.08) in the placebo group, which represented an overall treatment difference of −2.3 (p<0.001) (FIG. 1).

Statistically significant improvements in all three secondary efficacy endpoints were observed. At week 6, the mean (SD) change from baseline in MCC was 127.2 (139.3) mL in the BOTOX group compared with −1.8 (93.2) mL in the placebo group (p<0.001) (FIG. 3A). Treatment with BOTOX resulted in a statistically significant decrease (p=0.007) in MDP during the first IDC compared with the placebo group (−19.6 versus 3.7 cm H20) (FIG. 3B). Furthermore, a significant increase in the I-QOL total summary score at week 6 was observed with BOTOX treatment relative to placebo, which was approximately 3-fold greater than the minimally important difference (FIG. 4). Statistically significant improvements were also observed in other efficacy measures. Significantly higher proportions of patients in the BOTOX treatment group compared to placebo had reductions from baseline in urinary incontinence episodes at week 6 at all thresholds evaluated, including ≧50% (definition of responder) and 100% reduction (FIG. 2). Statistically significant increases in the BOTOX group compared to placebo were observed in volume per void at weeks 6 and 12.

Furthermore, statistically significant reductions in daily frequency of micturition episodes, urgency episodes, and nocturia episodes were observed in the BOTOX group compared with placebo at weeks 2, 6, and 12. A significantly (p<0.001) longer duration of effect as measured by the median time to request retreatment was observed in the BOTOX group compared with placebo (FIG. 6). Consequently, a significantly lower proportion of patients requested retreatment in the BOTOX group compared with placebo. For those patients who requested and received a second treatment (i.e., patients either received a second BOTOX injection or those receiving BOTOX for the first time), a similar magnitude of improvement in daily urinary incontinence episodes with BOTOX was observed in treatment cycle 2 compared to treatment cycle 1 with statistically significant changes from baseline within both treatment sequences at weeks 2, 6, and 12 (p<0.001). In addition, similar improvements with BOTOX were observed in secondary and other endpoints in treatment cycle 2 compared to treatment cycle 1.

For the I-QOL, significantly higher scores (improvements) for the total score and the 3 domains (“Avoidance and Limiting Behavior,” “Psychosocial Impact,” and “Social Embarrassment”) were observed in the BOTOX treatment group compared to the placebo group at week 6 (FIGS. 4 and 5). In addition, statistically significantly higher increases in OABPSTQ total summary score favoring BOTOX was observed compared with placebo at weeks 6 and 12. A significantly lower proportion of patients treated with BOTOX experienced an IDC at week 6 compared with placebo. In addition, in the minority of BOTOX-treatment patients who still had an IDC at week 6, a statistically significantly higher VP max IDC was observed in the BOTOX group compared with placebo (p<0.001). A statistically significant greater decrease from baseline was observed in the BOTOX group in Pdetmax compared with placebo at week 6 (p<0.001).

### Example 2

### Injection Paradigm Sub Study

A total of 41 patients were enrolled into an injection paradigm subset study, where 22 patients were treated with BOTOX and 19 treated with placebo, where the BOTOX or placebo was administered as 20 injections of 0.5 mL each. Three patients enrolled in the study (2 BOTOX, 1 placebo) discontinued prematurely. Overall, the baseline demographics and disease characteristics were similar between the two treatment groups.

The efficacy results in the injection paradigm subset study were similar and consistent with results from the study of Example 1, particularly for the BOTOX group. For the primary efficacy endpoint at week six, statistically significant decreases from baseline were observed in the BOTOX group compared to placebo (FIGS. 7A and 7B, FIG. 8). A similar magnitude of improvements were observed in the BOTOX group compared with BOTOX group in the main study for all three secondary efficacy endpoints (MCC, MDP at 1st IDC, and I-QOL total summary score) at week 6 (Table 1). The most common adverse events observed in the subset study (20 injections of 0.5 mL each) versus the main study (30 injections of 1 mL each) are reported in Table 2.

Table 1 compares the changes obtained from the 20 site injection paradigm versus the 30 site injection paradigm (main study of Example 1):

TABLE 1

| | Injection | |
|---|---|---|
| | Paradigm | |
| | Study | Main Study |
| | BOTOX 100 U | BOTOX 100 U |
| Endpoint | (Placebo) | (Placebo) |
| Mean Change from baseline to | N = 21/N = 19 | N = 62/N = 72 |
| week 6 in MCC (mL) | 174.9 ± 142.7 | 127.2 ± 139.3*** |
| | (80.6 ± 100.8) | (−1.8 ± 93.2) |
| Mean Change from baseline to | N = 11/N = 10 | N = 25/N = 51 |
| week 6 in MDP during 1^{st} IDC | −15.7 ± 28.6 | −19.6 ± 37.6** |
| (cm H₂O) | (14.9 ± 34.8) | (3.7 ± 33.2) |
| Mean Change from baseline to | N = 22/N = 19 | N = 66/N = 78 |
| week 6 in I-QOL Total Score | 33.6 ± 24.8 | 40.4 ± 26.5*** |
| | (18.0 ± 18.6) | (9.9 ± 15.9) |

Values shown are mean ± SD at Week 6

Table 2 presents a summary of commonly reported treatment adverse effects (TEAE) between a 20 site injection paradigm versus a 30 injection site injection paradigm.

TABLE 2
TEAEs >5% in BOTOX 100 U & Greater Than Placebo
Within the 1^{st} 12 Weeks of Treatment Cycle 1

| | Injection Paradigm Study | Main Study (Example 1) |
|---|---|---|
| | BOTOX 100 U (N = 22) | BOTOX 100 U (N = 66) |
| TEAE | (Placebo [N = 19]) | (Placebo [N = 78]) |
| Overall | 14 (63.6%) | 45 (68.2%) |
| | 6 (31.6%) | 38 (48.7%) |
| Disuria | 5 (22.7%) | 3 (4.5%) |
| | 1 (5.3%) | 1 (1.3%) |
| Urinary Retention | 4 (18.2%) | 10 (15.2%) |
| | 0 (0.0%) | 1 (1.3%) |
| Urinary Tract | 3 (13.6%) | 17 (25.8%) |
| Infection | 0 (0.0%) | 5 (6.4%) |
| Bacteriuria | 2 (9.1%) | 6 (9.1%) |
| | 1 (5.3%) | 4 (5.1%) |
| Pollakiuria | 2 (9.1%) | 0 (0.0%) |
| | 0 (0.0%) | 0 (0.0%) |
| Other AEs | | |
| of Interest | | |
| Haematuria | 0 (0.0%) | 2 (3.0%) |
| | 1 (5.3%) | 5 (6.4%) |

Many alterations and modifications may be made by those having ordinary skill in the art, without departing from the spirit and scope of the disclosure. Therefore, it must be understood that the described embodiments have been set forth only for the purposes of examples, and that the embodiments should not be taken as limiting the scope of the following claims. The following claims are, therefore, to be read to include not only the combination of elements which are literally set forth, but all equivalent elements for performing substantially the same function in substantially the same way to obtain substantially the same result. The claims are thus to be understood to include those that have been described above, those that are conceptually equivalent, and those that incorporate the ideas of the disclosure.

We claim:

1. A method for treating urinary incontinence associated with neurogenic detrusor overactivity in a patient in need thereof, comprising: locally administering to the bladder of the patient a composition comprising an amount of less than 200 Units a clostridial derivative.

2. The method of claim 1, wherein the clostridial derivative is a botulinum toxin.

3. The method of claim 2, wherein the botulinum toxin is selected from the group consisting of botulinum toxin types A, B, C, D, E, F and G.

4. The method of claim 3, wherein the botulinum neurotoxin is type A.

5. The method of claim 4, wherein the botulinum neurotoxin is onabotulinumtoxinA.

6. The method of claim 4, wherein the therapeutically effective amount is between about 10 Units to less than about 200 Units.

7. The method of claim 4, wherein the therapeutically effective amount is less than about 150 Units.

8. The method of claim 4, wherein the therapeutically effective amount is about 100 Units.

9. The method of claim 5, wherein the therapeutically effective amount is about 100 Units.

10. The method of claim 1, wherein the administering is by injection.

11. The method of claim 10, wherein the amount is administered at multiple injection sites.

12. The method of claim 11, wherein the multiple injection sites range from a number of injection sites between about 10 to 50 injection sites.

13. The method of claim 12, wherein the amount is administered in a number of injections, and wherein the number of injections is equal to the number of injection sites.

14. The method of claim 12, wherein the amount is administered in a number of injections, and wherein the number of injections is greater than the number of injection sites.

15. The method of claim 11, wherein the amount is administered in about 30 injection sites.

16. The method of claim 11, wherein the amount is administered in less than about 30 injection sites.

17. The method of claim 16, wherein the therapeutically effective amount is administered is administered in about 25 injection sites.

18. The method of claim 16, wherein the therapeutically effective amount is administered is administered in about 20 injection sites.

19. The method of claim 16, wherein the therapeutically effective amount is administered is administered in about 15 injection sites.

20. The method of claim 1, wherein said locally administering is to a patient having neurogenic-associated urinary incontinence wherein the neurogenic detrusor overactivity results from multiple sclerosis.

21. The method of claim 20, wherein said locally administering is to a patient who is not currently using clean intermittent catheterization (CIC).

22. The method of claim 20, wherein said locally administering is to a patient who is not responding or intolerant to anticholinergic medication.

23. A method for reducing or preventing the risk for urinary retention associated with neurogenic detrusor overactivity (NDO) treatment using a botulinum toxin in a patient, comprising: locally administering to the bladder of the patient a composition comprising an amount of the botululinum toxin type A, wherein the amount is less than 200 Units.

24. The method of claim 23, wherein the amount is about 100 Units.

25. The method of claim 23, wherein said locally administering is to a patient having neurogenic associated urinary incontinence, wherein the NDO results from multiple sclerosis.

26. The method of claim 25, wherein said locally administering is to a patient who is not currently using CIC.

27. The method of claim 23, wherein said locally administering is to a patient who is not responding or intolerant to anticholinergic medication.

28. A method for reducing clean intermittent catherization (CIC) associated with treatment of neurogenic detrusor overactivity (NDO) with a botulinum toxin in a patient, the method comprising: locally administering to the bladder of the patient a composition comprising a therapeutically effective amount of a botulinum toxin type A; wherein the therapeutically effective amount is less than 200 Units.

29. The method of claim 28, wherein the therapeutically effective amount is about 100 Units.

30. The method of claim 28, wherein said locally administering is to a patient having neurogenic associated urinary incontinence, wherein the NDO results from multiple sclerosis.

31. The method of claim 30, wherein said locally administering is to a patient who is not currently using CIC.

32. The method of claim 28, wherein said locally administering is to a patient who is not responding or is intolerant of anticholinergic medication.

33. A method for treating urinary incontinence associated with neurogenic detrusor overactivity in a patient in need thereof, the method comprising locally administering a composition comprising about 100 Units of a botulinum toxin A to the bladder of the patient, wherein the administration is by injection to about 20 injection sites.

34. A method for treating urinary incontinence associated with neurogenic detrusor overactivity in a patient in need thereof, the method comprising locally administering a composition comprising about 100 Units of a botulinum toxin A to the bladder of the patient, wherein the administration is by injection to about 30 injection sites.
